SEQUENCE LISTING (41 sequences)

SEQ ID NO: 1 - protein, 5 aa, synthetic construct
  REGION 1..5: note = Description of Artificial Sequence: Synthetic peptide
SYEMH

SEQ ID NO: 2 - protein, 5 aa, synthetic construct
  REGION 1..5: note = Description of Artificial Sequence: Synthetic peptide
HYGMN

SEQ ID NO: 3 - protein, 5 aa, synthetic construct
  REGION 1..5: note = Description of Artificial Sequence: Synthetic peptide
NSGMH

SEQ ID NO: 4 - protein, 5 aa, synthetic construct
  REGION 1..5: note = Description of Artificial Sequence: Synthetic peptide
NYYMY

SEQ ID NO: 5 - protein, 17 aa, synthetic construct
  REGION 1..17: note = Description of Artificial Sequence: Synthetic peptide
RINPSVGSTWYAQKFQG

SEQ ID NO: 6 - protein, 17 aa, synthetic construct
  REGION 1..17: note = Description of Artificial Sequence: Synthetic peptide
WVNTYTGEPTYADDFKG

SEQ ID NO: 7 - protein, 17 aa, synthetic construct
  REGION 1..17: note = Description of Artificial Sequence: Synthetic peptide
VIWYDGSKRYYADSVKG

SEQ ID NO: 8 - protein, 17 aa, synthetic construct
  REGION 1..17: note = Description of Artificial Sequence: Synthetic peptide
GINPSNGGTNFNEKFKN

SEQ ID NO: 9 - protein, 12 aa, synthetic construct
  REGION 1..12: note = Description of Artificial Sequence: Synthetic peptide
GKREGGTEYLRK

SEQ ID NO: 10 - protein, 9 aa, synthetic construct
  REGION 1..9: note = Description of Artificial Sequence: Synthetic peptide
EGEGLGFGD

SEQ ID NO: 11 - protein, 4 aa, synthetic construct
  REGION 1..4: note = Description of Artificial Sequence: Synthetic peptide
NDDY

SEQ ID NO: 12 - protein, 11 aa, synthetic construct
  REGION 1..11: note = Description of Artificial Sequence: Synthetic peptide
RDYRFDMGFDY

SEQ ID NO: 13 - protein, 12 aa, synthetic construct
  REGION 1..12: note = Description of Artificial Sequence: Synthetic peptide
RASQSVASSYLA

SEQ ID NO: 14 - protein, 16 aa, synthetic construct
  REGION 1..16: note = Description of Artificial Sequence: Synthetic peptide
RSSQSIVHSHGDTYLE

SEQ ID NO: 15 - protein, 11 aa, synthetic construct
  REGION 1..11: note = Description of Artificial Sequence: Synthetic peptide
RASQSVSSYLA

SEQ ID NO: 16 - protein, 15 aa, synthetic construct
  REGION 1..15: note = Description of Artificial Sequence: Synthetic peptide
RASKGVSTSGYSYLH

SEQ ID NO: 17 - protein, 7 aa, synthetic construct
  REGION 1..7: note = Description of Artificial Sequence: Synthetic peptide
GASNRHT

SEQ ID NO: 18 - protein, 7 aa, synthetic construct
  REGION 1..7: note = Description of Artificial Sequence: Synthetic peptide
KVSNRFS

SEQ ID NO: 19 - protein, 7 aa, synthetic construct
  REGION 1..7: note = Description of Artificial Sequence: Synthetic peptide
DASNRAT

SEQ ID NO: 20 - protein, 7 aa, synthetic construct
  REGION 1..7: note = Description of Artificial Sequence: Synthetic peptide
LASYLES

SEQ ID NO: 21 - protein, 8 aa, synthetic construct
  REGION 1..8: note = Description of Artificial Sequence: Synthetic peptide
QQYHNAIT

SEQ ID NO: 22 - protein, 9 aa, synthetic construct
  REGION 1..9: note = Description of Artificial Sequence: Synthetic peptide
FQGSHIPVT

SEQ ID NO: 23 - protein, 9 aa, synthetic construct
  REGION 1..9: note = Description of Artificial Sequence: Synthetic peptide
QQSSNWPRT

SEQ ID NO: 24 - protein, 9 aa, synthetic construct
  REGION 1..9: note = Description of Artificial Sequence: Synthetic peptide
QHSRDLPLT

SEQ ID NO: 25 - protein, 121 aa, synthetic construct
  REGION 1..121: note = Description of Artificial Sequence: Synthetic polypeptide
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSS

SEQ ID NO: 26 - protein, 118 aa, synthetic construct
  REGION 1..118: note = Description of Artificial Sequence: Synthetic polypeptide
EIQLVQSGAEVKKPGSSVKVSCKASGYTFTHYGMNWVRQAPGQGLEWVGVNNTYTGEPTYADDFKGRLTFTLDTSTSTAYMELSSLRSEDTAVYYCTREGEGLGFGDWGQGTTVTVSS

SEQ ID NO: 27 - protein, 112 aa, synthetic construct
  REGION 1..112: note = Description of Artificial Sequence: Synthetic polypeptide
QVQLVESGGGWQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 28 - protein, 120 aa, synthetic construct [sequence omitted]

SEQ ID NO: 29 - protein, 107 aa, synthetic construct
  REGION 1..107: note = Description of Artificial Sequence: Synthetic polypeptide
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIK

SEQ ID NO: 30 - protein, 112 aa, synthetic construct
  REGION 1..112: note = Description of Artificial Sequence: Synthetic polypeptide
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIK

SEQ ID NO: 31 - protein, 107 aa, synthetic construct [sequence omitted]

SEQ ID NO: 32 - protein, 111 aa, synthetic construct [sequence omitted]

SEQ ID NO: 33 - protein, 448 aa, synthetic construct
  REGION 1..448: note = Description of Artificial Sequence: Synthetic polypeptide
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 214 aa, synthetic construct
  REGION 1..214: note = Description of Artificial Sequence: Synthetic polypeptide
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 448 aa, synthetic construct
  REGION 1..448: note = Description of Artificial Sequence: Synthetic polypeptide
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 444 aa, synthetic construct
  REGION 1..444: note = Description of Artificial Sequence: Synthetic polypeptide
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPEAAGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 218 aa, synthetic construct
  REGION 1..218: note = Description of Artificial Sequence: Synthetic polypeptide
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 436 aa, synthetic construct
  REGION 1..436: note = Description of Artificial Sequence: Synthetic polypeptide
QVQLVESGGGWQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 214 aa, synthetic construct
  REGION 1..214: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 447 aa, synthetic construct
  REGION 1..447: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 218 aa, synthetic construct
  REGION 1..218: note = Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0150487 A1
(43) Pub. Date: May 9, 2024

(54) COMBINATIONS WITH AN ANTI HUMAN CD39 ANTIBODY, AN ANTI HUMAN PD-1 ANTIBODY, AND CHEMOTHERAPY IN GASTRIC CANCER

(71) Applicant: Trishula Therapeutics Inc., South San Francisco, CA (US)

(72) Inventors: Joyson Karakunnel, South San Francisco, CA (US); Achim Moesta, South San Francisco, CA (US); Courtney Beers, South San Francisco, CA (US); Alana Lerner, South San Francisco, CA (US); Benjamin Engelhardt, Ludwigshafen (DE); Akshanth R. Polepally, Danville, CA (US); Daniel E.H. Afar, San Jose, CA (US); Peter S. Moon, Agoura Hills, CA (US)

(21) Appl. No.: 18/179,057

(22) Filed: Mar. 6, 2023

Related U.S. Application Data
(60) Provisional application No. 63/316,726, filed on Mar. 4, 2022.
(60) Provisional application No. 63/328,475, filed on Apr. 7, 2022.

(51) Int. Cl. C07K 16/28; A61K 31/282; A61K 31/513; A61K 31/519; A61K 39/00; A61P 35/00

(52) U.S. Cl. CPC C07K 16/2896; A61K 31/282; A61K 31/513; A61K 31/519; A61P 35/00; C07K 16/2818; A61K 2039/507; A61K 2039/545

(57) ABSTRACT

Provided herein are dosing and combination therapies with a CD39 antibody, a PD-1 antibody, and FOLFOX or CAPDX in gastric cancer.

### PRIORITY CLAIM

This application claims the benefit of U.S. Provisional Application No. 63/316,726, filed Mar. 4, 2022 and U.S. Provisional Application No. 63/328,475, filed Apr. 7, 2022, which are incorporated by reference herein in their entirety.

### SEQUENCE LISTING

A computer readable form of the sequence listing in a ST26 .XML format, entitled “1107385.00074-Sequence-Listing.txt” created on or about Jul. 20, 2023 with a file size of about 57,000 bytes contains the nucleotide and amino acid sequences disclosed in this application and is hereby incorporated by reference in its entirety.

### FIELD

Provided herein are dosing and combination therapies with a CD39 antibody, a PD-1 antibody, and FOLFOX or CAPOX in gastric cancer.

### BACKGROUND

Immune checkpoint inhibitors have revolutionized the cancer treatment paradigm. These agents modulate the tumor microenvironment or other aspects of the immune system to overcome the immune suppression that a tumor elicits on the host immune system. Anti-PD-1/programmed cell death ligand-1 (PD-L1) agents target immunosuppressive pathways and have been shown to be beneficial in numerous tumor types.

Research over the past decade has led to the discovery of several new immunomodulatory pathways. Among them, the adenosine pathway has emerged as one of the most promising targets in immuno-oncology. Adenosine is an immunosuppressive metabolite produced at high levels within the tumor microenvironment. It is mainly generated extracellularly through sequential dephosphorylation of adenosine triphosphate (ATP) by the ectonucleotidases CD39 and CD73.

Reportedly, both CD39 and CD73 are elevated in blood neoplasias, such as leukemia and lymphoma, as well as in multiple solid tumor types. In solid tumors, ATP is abundantly released in the extracellular space, achieving concentrations more than a thousand times higher than in healthy tissues. This is primarily due to cell death in the tumor core, metabolic or hypoxic stress, and pro-inflammatory signals that stimulate active export of ATP.

Chemotherapy-induced immunogenic cell death is in part mediated by the release of extracellular ATP. Tumors are proficient at converting ATP into adenosine via CD39 and CD73 on malignant cells, regulatory immune cells, and the vasculature. These ectonucleotidases modulate purinergic signaling by scavenging mainly pro-inflammatory ATP and generating immunosuppressive adenosine.

CD39 is the main rate-limiting enzyme in the adenosine cascade and has an important role in tumor progression. Blockade of the CD39 enzymatic activity may stimulate antitumor immunity across a wide range of tumors by preventing the production of immunosuppressive adenosine and by promoting the accumulation of ATP in the tumor microenvironment. CD39 can, thus, be viewed as an immunological switch that shifts ATP-driven pro-inflammatory immune cell activity toward an anti-inflammatory state mediated by adenosine and is, therefore, a unique therapeutic target for oncology indications.

Gastroesophageal cancer (GEC) is the fifth most frequently diagnosed cancer and the third leading cause of cancer-related death worldwide. In 2018, more than 1.6 million new GEC cases and approximately 1.3 million associated deaths were estimated to occur. In the United States, an estimated 45,000 new cases of GEC will be diagnosed in 2019 and there will be approximately 27,000 GEC-related deaths. GEC remains difficult to cure because most patients present with advanced disease. The prognosis of advanced GEC is poor, with a 5-year overall survival (OS) rate estimated around 5% to 20%.

Treatment of advanced/metastatic GEC is based on human-epidermal growth factor receptor 2 (HER2) status. However, unlike in breast cancer, the prognostic significance of HER2 status in GEC is unclear. While further research is needed to assess the prognostic significance of HER2 status in GEC, the addition of HER2 monoclonal antibodies to chemotherapy regimens has proved to be a promising treatment option for patients with HER2-positive metastatic disease, which accounts for 12% to 23% of GEC.

For patients with HER2-negative GEC, chemotherapy is the standard first-line treatment for advanced disease. Currently, a fluoropyrimidine (fluorouracil or capecitabine) combined with a platinum (oxaliplatin or cisplatin) is the mainstay of chemotherapy.

A recent Phase 3 trial compared treatment with fluorouracil plus cisplatin to fluorouracil plus oxaliplatin (FOLFOX) in 220 patients with previously untreated advanced adenocarcinoma of the stomach or esophagogastric junction cancer. Results showed that FOLFOX was associated with significantly less toxicity and a trend toward improved median progression-free survival (PFS), compared with fluorouracil plus cisplatin (5.8 vs 3.9 months; p=0.77). In patients >65 years of age, FOLFOX resulted in superior response rates (41.3 vs 16.7%, respectively; p=0.12), time to treatment failure (5.4 vs 2.3 months; p<0.001), PFS (6.0 vs 3.1 months; p=0.029), and OS (13.9 vs 7.2 months), compared with fluorouracil plus cisplatin.

Anti-PD-1 agents are also evolving as front-line treatment for GEC. On 16 Apr. 2021, the US Food and Drug Administration approved a PD-1 antibody to be combined with select types of chemotherapy for initial treatment of patients with advanced or metastatic gastric cancer, gastroesophageal junction cancer, and esophageal adenocarcinoma. First-line treatment with a PD-1 antibody and chemotherapy demonstrated a statistically significant improvement in OS among previously untreated patients with PD-L1-positive advanced gastric cancer, gastroesophageal junction cancer, and esophageal adenocarcinoma compared to chemotherapy alone. The study demonstrated a statistically significant improvement in OS and PFS for patients with PD-L1 combined positive score (CPS)≥5.

While impressive and durable responses are observed in subsets of patients treated with anti-PD-1 agents, response rates are rarely >50%, and, for most patients, a response is transient. Thus, a synergistic strategy may be beneficial for patients. Co-expression of PD-1 and CD39 is highly prevalent on tumor-infiltrating lymphocytes and marks exhausted effector T-cell subsets in multiple tumor types. Chemotherapy remains a key therapeutic option for many cancer patients and it has been conventionally believed to directly kill tumor cells through their cytotoxic effects and trigger tumor cell death in a nonimmunogenic manner.

What is needed are combination therapies.

### SUMMARY

Disclosed herein are dosing and combination therapies with a CD39 antibody, a PD-1 antibody, and FOLFOX or CAPOX.

A first aspect provides a method for treating a subject suffering from gastric cancer, comprising the step of administering to the subj ect a pharmaceutical composition comprising:
i) an anti-human CD39 antibody comprising an antibody comprising a heavy chain variable region (VH) and a light chain variable region (VL), wherein the VH comprises or consists of:
a) a VHCDR1 having the sequence set forth in SEQ ID NO: 1,
b) a VHCDR2 having the sequence set forth in SEQ ID NO: 5, and
c) a VHCDR3 having the sequence set forth in SEQ ID NO: 9, and wherein the VL comprises or consists of:
a) a VLCDR1 having the sequence set forth in SEQ ID NO: 13,
b) a VLCDR2 having the sequence set forth in SEQ ID NO: 17, and
c) a VLCDR3 having the sequence set forth in SEQ ID NO: 21;
ii) an anti-human PD-1 antibody comprising an antibody comprising a heavy chain variable region (VH) and a light chain variable region (VL), wherein the VH comprises or consists of:
a) a VHCDR1 having the sequence set forth in SEQ ID NO: 2,
b) a VHCDR2 having the sequence set forth in SEQ ID NO: 6, and
c) a VHCDR3 having the sequence set forth in SEQ ID NO: 10, and wherein the VL comprises or consists of:
a) a VLCDR1 having the sequence set forth in SEQ ID NO: 14,
b) a VLCDR2 having the sequence set forth in SEQ ID NO: 18, and
c) a VLCDR3 having the sequence set forth in SEQ ID NO: 22; and
iii) FOLFOX or CAPOX.

A second aspect provide aspect provides a pharmaceutical composition for treating a subject suffering from gastric cancer, the pharmaceutical composition comprising or consisting of:
i) i) an anti-human CD39 antibody comprising an antibody comprising a heavy chain variable region (VH) and a light chain variable region (VL), wherein the VH comprises or consists of:
a) a VHCDR1 having the sequence set forth in SEQ ID NO: 1,
b) a VHCDR2 having the sequence set forth in SEQ ID NO: 5, and
c) a VHCDR3 having the sequence set forth in SEQ ID NO: 9, and wherein the VL comprises or consists of:
a) a VLCDR1 having the sequence set forth in SEQ ID NO: 13,
b) a VLCDR2 having the sequence set forth in SEQ ID NO: 17, and
c) a VLCDR3 having the sequence set forth in SEQ ID NO: 21;
ii) an anti-human PD-1 antibody comprising an antibody comprising a heavy chain variable region (VH) and a light chain variable region (VL), wherein the VH comprises or consists of:
a) a VHCDR1 having the sequence set forth in SEQ ID NO: 2,
b) a VHCDR2 having the sequence set forth in SEQ ID NO: 6, and
c) a VHCDR3 having the sequence set forth in SEQ ID NO: 10, and wherein the VL comprises or consists of:
a) a VLCDR1 having the sequence set forth in SEQ ID NO: 14,
b) a VLCDR2 having the sequence set forth in SEQ ID NO: 18, and
c) a VLCDR3 having the sequence set forth in SEQ ID NO: 22; and
iii) FOLFOX or CAPOX.

In some embodiments, the anti-human CD39 antibody is administered at a dosage of from 0.5 mg/kg to 40.0 mg/kg. In some embodiments, the anti-human CD39 antibody is administered at a dosage of 20.0 mg/kg.

In some embodiments, the anti-human PD-1 antibody is administered at a dosage of 500 mg. In some embodiments, FOLFOX is administered with components having a dosage in the following amounts: oxaliplatin at 85 mg/m²; leucovarin at 400 mg/m²; and 5-FU at a bolus infusion of 400 mg/m² and a continuous infusion of 2400 mg/m². In some embodiments, FOLFOX comprises mFOLFOX6.

In some embodiments, the gastric cancer comprises locally advanced or metastatic gastric cancer. In some embodiments, the gastric cancer is Her2⁻.

In some embodiments, the step of administering any of the antibodies or FOLFOX or CAPOX comprises intravenous administration. In some embodiments, the pharmaceutical composition may be administered intravenously.

In some embodiments, a single loading dose of the anti-human CD39 antibody is administered to the subject at a dosage of 40.0 mg/kg. In some embodiments, one week after administering the loading dose, the anti-human CD39 antibody is administered to the subject at a dosage of 20.0 mg/kg every two weeks.

In some embodiments, the anti-human PD-1 antibody is administered to the subject at a dosage of 500 mg every four weeks. In some embodiments, FOLFOX comprises mFOLFOX6 and the mFOLFOX6 is administered every 2 weeks.

In some embodiments, the anti-human CD39 antibody is an antibody comprising a heavy chain variable region (VH) and a light chain variable region (VL), the heavy chain variable region comprising or consisting of a molecule having a sequence comprising or consisting of SEQ ID NO: 25 and the light chain variable region comprising or consisting of a molecule having a sequence comprising or consisting of SEQ ID NO: 29. In some embodiments, the anti-human CD39 antibody is an antibody comprising a heavy chain and a light chain, the heavy chain comprising or consisting of one or more molecules having a sequence comprising or consisting of SEQ ID NO: 33 and the light chain comprising or consisting of one or more molecules having a sequence comprising or consisting of SEQ ID NO: 34.

In some embodiments, the anti-human PD-1 antibody is an antibody comprising a heavy chain variable region (VH) and a light chain variable region (VL), the heavy chain variable region comprising or consisting of a molecule having a sequence comprising or consisting of SEQ ID NO: 26 and the light chain variable region comprising or consisting of a molecule having a sequence comprising or consisting of SEQ ID NO: 30. In some embodiments, the anti-human PD-1 antibody is an antibody comprising a heavy chain and a light chain, the heavy chain comprising or consisting of one or more molecules having a sequence comprising or consisting of SEQ ID NO: 35 or SEQ ID NO: 36 and the light chain comprising or consisting of one or more molecules having a sequence comprising or consisting of SEQ ID NO: 37.

In some embodiments, the subject has a Partial Response or a Complete Response. In some embodiments, the method or pharmaceutical composition produces an OS of 40%, 50%, 60%, 70%, or 80%. In some embodiments, the method or pharmaceutical composition increases the PFS or DFS.

### BRIEF DESCRIPTION OF DRAWINGS

FIG. 1 shows CD39 monotherapy dose escalation and expansion.

FIG. 2 shows eligibility and patient demographics and disease characteristics for treatment with a CD39 antibody, an anti-PD-1 antibody (budigalimab), and FOLFOX for metastatic gastroesophageal cancer.

FIG. 3A shows patient response after treatment with a CD39 antibody, budigalimab, and FOLFOX for metastatic gastroesophageal cancer, specifically a treatment duration swim plot.

FIG. 3B shows patient response after treatment with a CD39 antibody, budigalimab, and FOLFOX for metastatic gastroesophageal cancer, specifically swimlane by CPS score.

FIG. 4 shows maximum change from baseline for treatment with a CD39 antibody, budigalimab, and FOLFOX for metastatic gastroesophageal cancer.

FIG. 5 shows onset of tumor responses for treatment with a CD39 antibody, budigalimab, and FOLFOX for metastatic gastroesophageal cancer.

FIGS. 6A shows combination treatment leads to an increase in PD-L1 CPS score and CD8 T cells.

FIG. 6B shows combination treatment leads to an increase in CD8 cells.

FIG. 6C shows combination treatment impacts CD39 stromal treatment.

### DESCRIPTION

### 1. Definitions

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

The terms “CD39” and “CD39 antigen” and “Cluster of Differentiation 39” are used interchangeably herein. CD39 is also known as also known as ectonucleoside triphosphate diphosphohydrolase-1 (gene: ENTPDJ; protein: NTPDase1, See www.ncbi.nlm.nih.gov/gene/953). CD39 has also been referred to as ATPDase and SPG64.

The terms “PD-1,” “programmed cell death protein 1,” and “Cluster of Differentiation 279” are used interchangeably herein.

As used herein, “FOLFOX” shall refer to a combination chemotherapy that includes leucovorin, fluorouracil, and oxalipiatin. There are several different FOLFOX regimens, including FOLFOX-4, FOLFOX-6, modified FOLFOX-6 (inFOLFOX-6), and FOLFOX-7. They differ in the doses and ways in which the three drugs are given.

As used herein, “CAPOX” refers to a combination chemotherapy that includes oxaliplatin and capecitabine. CAPOX may also be referred to as XELOX, CAPE-OX, and OxCap.

As used herein, “RP2D” refers to the recommend phase 2 dose of an agent or agents determined by dose-limiting toxicity. In oncology, it is defined as the dose level producing around 20% of dose-limiting toxicity. In North America, the Maximum Tolerated Dose is the RP2D whereas in the rest of the world the MTD is considered a dose greater than the RP2D. An RPTD is often chosen by the sponsor in consultation with investigators for dose expansion arms based on safety, tolerability, efficacy, pharmacokinetic, and pharmacokinetic data collected during the dose escalation portion of the study.

As used herein, “Overall Survival” or “OS” shall refer to the time which begins at diagnosis (or at the start of treatment) and ends at death.

As used herein, “Disease Free Survival” or “DFS” or “Relapse Free Survival” or “RFS” refers to the length of time after the treatment for cancer ended that the patient survives without any signs or symptoms of cancer.

As used herein, “Progression Free Survival” or “PFS” refers to the time from the participant's first dose of study treatment to the first date of either disease progression or death, whichever occurs first.

“Duration of Response” and “DOR” shall refer to the time from first documentation of disease response (CR or PR) until first documentation of progression or death from any cause, whichever occurs first.

“Objective Response Rate” or “ORR” refers to the percentage of subjects with unconfirmed and confirmed CR or unconfirmed and confirmed PR.

“Complete Response” or “CR” refers to the disappearance of all target lesions; any pathologic lymph nodes (whether target or nontarget lesions) must have reduction in short axis to less than 10 mm.

“Partial Response” or “PR” refers to at least 30% decrease in the sum of diameters of target lesions; reference is the baseline diameters.

As used herein, “Stable Disease” of “SD” shall refer to neither sufficient shrinkage compared to baseline to qualify for partial response nor sufficient increase (taking as reference the smallest sum diameters while on study) to qualify for PD.

As used herein, “Progressive Disease” or “PD” shall refer to at least a 20% increase in the sum of diameters of target lesions, taking as reference the smallest sum on study (this includes the baseline sum if that is the smallest sum on study). In addition to the relative increase of 20%, the sum must also demonstrate an absolute increase of at least 5 mm. The appearance of one or more new lesions is also considered progression.

As used herein, “DCR,” “Disease Control Rate,” “CBR,” and “Clinical Benefit Rate” shall refer to the percentage of patients with advanced or metastatic cancer who have achieved CR, PR, and SD.

As used herein, “CPS” shall refer to the total number of PD-L1 staining cells (tumor cells, lymphocytes, macrophages) divided by the total number of viable tumor cells multiplied by 100.

As used herein, “BOR” or “Best Overall Response” shall refer to the single best response status at any evaluation assessment timepoint prior to receipt of non-protocol therapy or prior to PD.

As used herein, the term “subject” means a mammalian subject. In some embodiments, the subject is a human.

### 2. Method(s)

Disclosed herein are combination and dosing regimens and compositions combining a CD39 antibody, a PD-1 antibody, and FOLFOX or CAPOX.

A first aspect provides a method for treating a subject suffering from gastric cancer, comprising the step of administering to the subject a pharmaceutical composition comprising:
i) an anti-human CD39 antibody comprising an antibody comprising a heavy chain variable region (VH) and a light chain variable region (VL), wherein the VH comprises or consists of:
a) a VHCDR1 having the sequence set forth in SEQ ID NO: 1,
b) a VHCDR2 having the sequence set forth in SEQ ID NO: 5, and
c) a VHCDR3 having the sequence set forth in SEQ ID NO: 9, and wherein the VL comprises or consists of:
a) a VLCDR1 having the sequence set forth in SEQ ID NO: 13,
b) a VLCDR2 having the sequence set forth in SEQ ID NO: 17, and
c) a VLCDR3 having the sequence set forth in SEQ ID NO: 21;
ii) an anti-human PD-1 antibody comprising an antibody comprising a heavy chain variable region (VH) and a light chain variable region (VL), wherein the VH comprises or consists of:
a) a VHCDR1 having the sequence set forth in SEQ ID NO: 2,
b) a VHCDR2 having the sequence set forth in SEQ ID NO: 6, and
c) a VHCDR3 having the sequence set forth in SEQ ID NO: 10, and wherein the VL comprises or consists of:
a) a VLCDR1 having the sequence set forth in SEQ ID NO: 14,
b) a VLCDR2 having the sequence set forth in SEQ ID NO: 18, and
c) a VLCDR3 having the sequence set forth in SEQ ID NO: 22; and
iii) FOLFOX or CAPOX.

In some embodiments, the anti-human CD39 antibody is administered at a dosage of from 0.5 mg/kg to 40.0 mg/kg. In some embodiments, the anti-human CD39 antibody is administered at a dosage of 20.0 mg/kg.

In some embodiments, the anti-human PD-1 antibody is administered at a dosage of 500 mg. In some embodiments, FOLFOX is administered with components having a dosage in the following amounts: oxaliplatin at 85 mg/m²; leucovarin at 400 mg/m²; and 5-FU at a bolus infusion of 400 mg/m² and a continuous infusion of 2400 mg/m². In some embodiments, FOLFOX comprises mFOLFOX6.

In some embodiments, the gastric cancer comprises locally advanced or metastatic gastric cancer. In some embodiments, the gastric cancer is Her2″.

In some embodiments, the step of administering any of the antibodies and/or FOLFOX or CAPOX comprises intravenous administration. In some embodiments, the pharmaceutical composition may be administered intravenously.

In some embodiments, a single loading dose of the anti-human CD39 antibody is administered to the subject at a dosage of 40.0 mg/kg. In some embodiments, one week after administering the loading dose, the anti-human CD39 antibody is administered to the subject at a dosage of 20.0 mg/kg every two weeks.

In some embodiments, the anti-human PD-1 antibody is administered to the subject at a dosage of 500 mg every four weeks. In some embodiments, FOLFOX comprises mFOLFOX6 and the mFOLFOX6 is administered every 2 weeks.

In some embodiments, the anti-human CD39 antibody is an antibody comprising a heavy chain variable region (VH) and a light chain variable region (VL), the heavy chain variable region comprising or consisting of a molecule having a sequence comprising or consisting of SEQ ID NO: 25 and the light chain variable region comprising or consisting of a molecule having a sequence comprising or consisting of SEQ ID NO: 29. In some embodiments, the anti-human CD39 antibody is an antibody comprising a heavy chain and a light chain, the heavy chain comprising or consisting of one or more molecules having a sequence comprising or consisting of SEQ ID NO: 33 and the light chain comprising or consisting of one or more molecules having a sequence comprising or consisting of SEQ ID NO: 34.

In some embodiments, the anti-human PD-1 antibody is budigalimab comprising a heavy chain variable region (VH) and a light chain variable region (VL), the heavy chain variable region comprising or consisting of a molecule having a sequence comprising or consisting of SEQ ID NO: 26 and the light chain variable region comprising or consisting of a molecule having a sequence comprising or consisting of SEQ ID NO: 30. In some embodiments, budigalimab comprises a heavy chain and a light chain, the heavy chain comprising or consisting of one or more molecules having a sequence comprising or consisting of SEQ ID NO: 35 or SEQ ID NO: 36 and the light chain comprising or consisting of one or more molecules having a sequence comprising or consisting of SEQ ID NO: 37.

Other anti-human PD-1 antibodies may be used. For example, in some embodiments, the anti-human PD-1 antibody is nivolumab and comprises or consists of an antibody comprising a heavy chain variable region (VH) and a light chain variable region (VL), wherein the VH comprises or consists of:
a) a VHCDR1 having the sequence set forth in SEQ ID NO: 3,
b) a VHCDR2 having the sequence set forth in SEQ ID NO: 7, and
c) a VHCDR3 having the sequence set forth in SEQ ID NO: 11, and wherein the VL comprises or consists of:
a) a VLCDR1 having the sequence set forth in SEQ ID NO: 15,
b) a VLCDR2 having the sequence set forth in SEQ ID NO: 19, and
c) a VLCDR3 having the sequence set forth in SEQ ID NO: 23.

In some embodiments, the anti-human PD-1 antibody is an antibody comprising a heavy chain variable region (VH) and a light chain variable region (VL), the heavy chain variable region comprising or consisting of a molecule having a sequence comprising or consisting of SEQ ID NO: 27 and the light chain variable region comprising or consisting of a molecule having a sequence comprising or consisting of SEQ ID NO: 31. In some embodiments, the anti-human PD-1 antibody is an antibody comprising a heavy chain and a light chain, the heavy chain comprising or consisting of one or more molecules having a sequence comprising or consisting of SEQ ID NO: 38 and the light chain comprising or consisting of one or more molecules having a sequence comprising or consisting of SEQ ID NO: 39.

In some embodiments, the anti-human PD-1 antibody is pembrolizamab and comprises or consists of an antibody comprising a heavy chain variable region (VH) and a light chain variable region (VL), wherein the VH comprises or consists of:
a) a VHCDR1 having the sequence set forth in SEQ ID NO: 4,
b) a VHCDR2 having the sequence set forth in SEQ ID NO: 8, and
c) a VHCDR3 having the sequence set forth in SEQ ID NO: 12, and wherein the VL comprises or consists of:
a) a VLCDR1 having the sequence set forth in SEQ ID NO: 16,
b) a VLCDR2 having the sequence set forth in SEQ ID NO: 20, and
c) a VLCDR3 having the sequence set forth in SEQ ID NO: 24.

In some embodiments, the anti-human PD-1 antibody is an antibody comprising a heavy chain variable region (VH) and a light chain variable region (VL), the heavy chain variable region comprising or consisting of a molecule having a sequence comprising or consisting of SEQ ID NO: 28 and the light chain variable region comprising or consisting of a molecule having a sequence comprising or consisting of SEQ ID NO: 32. In some embodiments, the anti-human PD-1 antibody is an antibody comprising a heavy chain and a light chain, the heavy chain comprising or consisting of one or more molecules having a sequence comprising or consisting of SEQ ID NO: 40 and the light chain comprising or consisting of one or more molecules having a sequence comprising or consisting of SEQ ID NO: 41.

### 3. Pharmaceutical Compositions

A second aspect provide aspect provides a pharmaceutical composition for treating a subject suffering from gastric cancer, the pharmaceutical composition comprising or consisting of:
i) an anti-human CD39 antibody comprising an antibody comprising a heavy chain variable region (VH) and a light chain variable region (VL), wherein the VH comprises or consists of:
a) a VHCDR1 having the sequence set forth in SEQ ID NO: 1,
b) a VHCDR2 having the sequence set forth in SEQ ID NO: 5, and
c) a VHCDR3 having the sequence set forth in SEQ ID NO: 9, and wherein the VL comprises or consists of:
a) a VLCDR1 having the sequence set forth in SEQ ID NO: 13,
b) a VLCDR2 having the sequence set forth in SEQ ID NO: 17, and
c) a VLCDR3 having the sequence set forth in SEQ ID NO: 21;
ii) an anti-human PD-1 antibody comprising an antibody comprising a heavy chain variable region (VH) and a light chain variable region (VL), wherein the VH comprises or consists of:
a) a VHCDR1 having the sequence set forth in SEQ ID NO: 2,
b) a VHCDR2 having the sequence set forth in SEQ ID NO: 6, and
c) a VHCDR3 having the sequence set forth in SEQ ID NO: 10, and wherein the VL comprises or consists of:
a) a VLCDR1 having the sequence set forth in SEQ ID NO: 14,
b) a VLCDR2 having the sequence set forth in SEQ ID NO: 18, and
c) a VLCDR3 having the sequence set forth in SEQ ID NO: 22; and
iii) FOLFOX or CAPOX.

Pharmaceutical compositions for each of the respective antibodies and FOLFOX or CAPOX will contain one or more active agents and may further contain solvents, buffers, diluents, carriers, and other excipients to aid the administration, solubility, absorption or bioavailability, and/or stability, etc. of the active agent(s) or overall composition. Each of the antibodies and FOLFOX or CAPOX can be provided in an appropriate pharmaceutical composition and administered by any suitable route of administration. Suitable routes of administration for each of the antibodies and FOLFOX or CAPOX include, but are not limited to, inhalation, intraarterial, intradermal, intramuscular, intraperitoneal, intravenous, nasal, parenteral, pulmonary, and subcutaneous routes. Each composition may comprise one or more pharmaceutical excipients. Any suitable pharmaceutical excipient may be used and one of ordinary skill in the art is capable of selecting suitable pharmaceutical excipients. Additional pharmaceutical excipients include, for example, those described in the Handbook of Pharmaceutical Excipients, Rowe et al. (Eds.) 6th Ed. (2009), incorporated by reference in its entirety.

### 4. Dosage Forms

The doctor will determine the dosology which she considers most appropriate according to age, weight, condition, and other factors specific to the subject to be treated, including available guidance on recommend dosages.

In some embodiments, the anti human CD39 antibody is administered at a dosage from 0.5 mg/kg to 60 mg/kg. In some embodiments, the anti human CD39 antibody is administered at a dosage from 0.5 mg/kg to 1 mg/kg. In some embodiments, the anti human CD39 antibody is administered at a dosage from 1 mg/kg to 5 mg/kg. In some embodiments, the anti human CD39 antibody is administered at a dosage from 5 mg/kg to 10 mg/kg. In some embodiments, the anti human CD39 antibody is administered at a dosage from 10 mg/kg to 15 mg/kg. In some embodiments, the anti human CD39 antibody is administered at a dosage from 15 mg/kg to 20 mg/kg. In some embodiments, the anti human CD39 antibody is administered at a dosage from 20 mg/kg to 25 mg/kg. In some embodiments, the anti human CD39 antibody is administered at a dosage from 25 mg/kg to 30 mg/kg. In some embodiments, the anti human CD39 antibody is administered at a dosage from 30 mg/kg to 35 mg/kg. In some embodiments, the anti human CD39 antibody is administered at a dosage from 35 mg/kg to 40 mg/kg. In some embodiments, the anti human CD39 antibody is administered at a dosage from 40 mg/kg to 45 mg/kg. In some embodiments, the anti human CD39 antibody is administered at a dosage from 45 mg/kg to 50 mg/kg. In some embodiments, the anti human CD39 antibody is administered at a dosage from 50 mg/kg to 55 mg/kg. In some embodiments, the anti human CD39 antibody is administered at a dosage from 55 mg/kg to 60 mg/kg.

In some embodiments, the anti human CD39 antibody is administered at a dosage of 30 mg/kg Q3W or a dosage of 20 mg/kg Q2W.

In some embodiments, the anti human PD-1 antibody is administered at a dosage from 1 mg to 1 g. In some embodiments, the anti human PD-1 antibody is administered at a dosage of 50 mg. In some embodiments, the anti human PD-1 antibody is administered at a dosage of 100 mg. In some embodiments, the anti human PD-1 antibody is administered at a dosage of 150 mg. In some embodiments, the anti human PD-1 antibody is administered at a dosage of 200 mg. In some embodiments, the anti human PD-1 antibody is administered at a dosage of 250 mg. In some embodiments, the anti human PD-1 antibody is administered at a dosage of 300 mg. In some embodiments, the anti human PD-1 antibody is administered at a dosage of 350 mg. In some embodiments, the anti human PD-1 antibody is administered at a dosage of 400 mg. In some embodiments, the anti human PD-1 antibody is administered at a dosage of 450 mg. In some embodiments, the anti human PD-1 antibody is administered at a dosage of 500 mg. In some embodiments, the anti human PD-1 antibody is administered at a dosage of 550 mg. In some embodiments, the anti human PD-1 antibody is administered at a dosage of 600 mg. In some embodiments, the anti human PD-1 antibody is administered at a dosage of 650 mg. In some embodiments, the anti human PD-1 antibody is administered at a dosage of 700 mg. In some embodiments, the anti human PD-1 antibody is administered at a dosage of 750 mg. In some embodiments, the anti human PD-1 antibody is administered at a dosage of 800 mg. In some embodiments, the anti human PD-1 antibody is administered at a dosage of 850 mg. In some embodiments, the anti human PD-1 antibody is administered at a dosage of 900 mg. In some embodiments, the anti human PD-1 antibody is administered at a dosage of 950 mg.

In some embodiments, the anti human PD-1 antibody is administered at a flat dose of 275 mg Q2W, a flat dose of 375 mg Q3W, or a flat dose of 500 mg Q4W.

In some embodiments, treatment or prevention can be initiated with one or more loading doses of an antibody or composition provided herein followed by one or more maintenance doses. In some embodiments, the one or more loading doses comprise one or more loading doses of the anti human CD39 antibody. In some embodiments, the one or more loading doses is administered at a dosage of between 5 mg/kg and 100 mg/kg. In some embodiments, a single loading dose of the anti-human CD39 antibody is administered to the subject at a dosage of 5.0 mg/kg. In some embodiments, a single loading dose of the anti-human CD39 antibody is administered to the subject at a dosage of 10.0 mg/kg. In some embodiments, a single loading dose of the anti-human CD39 antibody is administered to the subject at a dosage of 15.0 mg/kg. In some embodiments, a single loading dose of the anti-human CD39 antibody is administered to the subject at a dosage of 20.0 mg/kg. In some embodiments, a single loading dose of the anti-human CD39 antibody is administered to the subject at a dosage of 25.0 mg/kg. In some embodiments, a single loading dose of the anti-human CD39 antibody is administered to the subject at a dosage of 30.0 mg/kg. In some embodiments, a single loading dose of the anti-human CD39 antibody is administered to the subject at a dosage of 35.0 mg/kg. In some embodiments, a single loading dose of the anti-human CD39 antibody is administered to the subject at a dosage of 40.0 mg/kg. In some embodiments, a single loading dose of the anti-human CD39 antibody is administered to the subject at a dosage of 45.0 mg/kg. In some embodiments, a single loading dose of the anti-human CD39 antibody is administered to the subject at a dosage of 50.0 mg/kg. In some embodiments, a single loading dose of the anti-human CD39 antibody is administered to the subject at a dosage of 55.0 mg/kg. In some embodiments, a single loading dose of the anti-human CD39 antibody is administered to the subject at a dosage of 60.0 mg/kg. In some embodiments, a single loading dose of the anti-human CD39 antibody is administered to the subject at a dosage of 65.0 mg/kg. In some embodiments, a single loading dose of the anti-human CD39 antibody is administered to the subject at a dosage of 70.0 mg/kg. In some embodiments, a single loading dose of the anti-human CD39 antibody is administered to the subject at a dosage of 75.0 mg/kg. In some embodiments, a single loading dose of the anti-human CD39 antibody is administered to the subject at a dosage of 80.0 mg/kg. In some embodiments, a single loading dose of the anti-human CD39 antibody is administered to the subject at a dosage of 85.0 mg/kg. In some embodiments, a single loading dose of the anti-human CD39 antibody is administered to the subject at a dosage of 90.0 mg/kg. In some embodiments, a single loading dose of the anti-human CD39 antibody is administered to the subject at a dosage of 95.0 mg/kg.

In some embodiments, FOLFOX is administered with components having a dosage in the following amounts: oxaliplatin at 85 mg/m²; leucovarin at 400 mg/m²; and 5-FU at a bolus infusion of 400 mg/m² and a continuous infusion of 2400 mg/m². In some embodiments, FOLFOX comprises mFOLFOX6.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

### 5. Scheduling

The frequency and dosage will also vary according to factors specific for each subject depending on the specific therapy administered as well as age, body, weight, response, and the past medical history of the subject. Furthermore, it is noted that the clinician or treating physician will know how and when to interrupt, adjust, or terminate therapy in conjunction with subject response.

In some embodiments, the combination is assembled in the subject being treated, whether they are administered at the same time or different times. In some embodiments, the components of the combination are mixed together at the point of care prior to administration.

In some embodiments, the CD39 antibody, PD-1 antibody, and FOLFOX or CAPOX are administered on the same schedule. In some embodiments, the CD39 antibody, PD-1 antibody, and FOLFOX or CAPOX are administered on a different schedule. In some embodiments, the CD39 antibody and PD-1 antibody are administered on the same schedule but the FOLFOX or CAPOX are administered on a different schedule.

In some embodiments, the CD39 antibody is administered once a week. In some embodiments, the CD39 antibody is administered every other week. In some embodiments, the CD39 antibody is administered every three weeks. In some embodiments, the CD39 antibody is administered every four weeks. In some embodiments, the CD39 antibody is administered every five weeks. In some embodiments, the CD39 antibody is administered every six weeks. In some embodiments, the CD39 antibody is administered every seven weeks. In some embodiments, the CD39 antibody is administered every eight weeks.

In some embodiments, the dose and dosing frequency of the CD39 antibody is 20 mg/kg every two weeks. In some embodiments, the dose and dosing frequency of the CD39 antibody is 30 mg/kg every three weeks. In some embodiments, the dose and dosing frequency of the CD39 antibody is 40 mg/kg every four weeks.

In some embodiments, the PD-1 antibody is administered once a week. In some embodiments, the PD-1 antibody is administered every other week. In some embodiments, the PD-1 antibody is administered every three weeks. In some embodiments, the PD-1 antibody is administered every four weeks. In some embodiments, the CD39 antibody is administered every five weeks. In some embodiments, the PD-1 antibody is administered every six weeks. In some embodiments, the PD-1 antibody is administered every seven weeks. In some embodiments, the PD-1 antibody is administered every eight weeks.

In some embodiments, the dose and dosing frequency of the PD-1 antibody is between 100 mg and 300 mg every week. In some embodiments, the dose and dosing frequency of the PD-1 antibody is between 200 mg and 400 mg every two weeks. In some embodiments, the dose and dosing frequency of the PD-1 antibody is 300 mg and 500 mg every three weeks. In some embodiments, the dose and dosing frequency of the PD-1 antibody is 400 mg and 600 mg every four weeks. In some embodiments, the dose and dosing frequency of the PD-1 antibody is 500 mg and 700 mg every five weeks. In some embodiments, the dose and dosing frequency of the PD-1 antibody is 600 mg and 800 mg every six weeks. In some embodiments, the dose and dosing frequency of the PD-1 antibody is 700 mg and 900 mg every seven weeks. In some embodiments, the dose and dosing frequency of the PD-1 antibody is 800 mg and 1 g every eight weeks.

In some embodiments, the anti-human CD39 antibody is administered on days 1 and 15 of a 28 day cycle. In some embodiments, the anti-human PD1 antibody is administered to the subject at a dosage of 500 mg every four weeks. In some embodiments, mFOLFOX6 is administered on days 1 and 15 of a 28 day cycle.

In some embodiments, the dosing regimen further comprises an initial loading dose of 40 mg/kg, one week before beginning a repetitive portion of a dosing regimen (maintenance doses).

In some embodiments, the dosing regimen further comprises an initial loading dose between 10 mg/kg and 90 mg/kg. In some embodiments, the loading dose is 10 mg/kg. In some embodiments, the loading dose is 20 mg/kg. In some embodiments, the loading dose is 30 mg/kg. In some embodiments, the loading dose is 40 mg/kg. In some embodiments, the loading dose is 50 mg/kg. In some embodiments, the loading dose is 60 mg/kg. In some embodiments, the loading dose is 70 mg/kg. In some embodiments, the loading dose is 80 mg/kg. In some embodiments, the loading dose is 90 mg/kg.

These dosage regimens are generally sufficient to maintain the trough concentration of the anti-human CD39 antibody at or above 50 μg/ml. Other trough concentrations are achievable by adjusting the dosage accordingly. These particular regimens offer the benefit of relatively convenient out-patient treatment, but other dosing regimens involving smaller dosages administered more frequently can also be used, up to and including a continuous intravenous infusion (IV drip). Any of these dosing regimens can be referred to as a means or step for maintaining the serum trough concentration of the CD39 binding protein at or above a threshold level—or at or above a threshold level in 85% of subjects. In some embodiments, the threshold level is 10, 20, 30, 40, 50, or 60 μg/mL.

### 6. RECIST and Response

Assessment of tumor burden is an important criteria of evaluation. Any method can be used to assess response. Some embodiments provide for assessing response using RECIST criteria (See, https://recist.eortc.org/recist-1-1-2/ and New response evaluation criteria in solid tumors: Revised RECIST guideline (version 1.1), European Journal of Cancer 45 (2009) 228-247, incorporated by reference in their entirety herein).

To demonstrate efficacy, subjects treated with the method or compositions should have an objective response rate greater than 25%, or greater than 30%. In some embodiments, efficacy of the treatment includes a median DOR of 6 months or more (i.e., of at least 6 months, at least 8 months, and/or at least 10 months). Other efficacy endpoints include PFS, OS, and/or an acceptable safety and tolerability profile.

In some embodiments, the method or pharmaceutical compositions provides a Partial Response or a Complete Response. In some embodiments, the method or pharmaceutical composition produces an OS of 20%, 25%, 30%, 35%, 40%, 45%, 50%, 55%, 60%, 65%, 70%, 75%, 80%, 85%, or 90%. In some embodiments, the method or pharmaceutical composition increases the PFS or DFS.

Different methods may be used to measure a tumor. Some internal cancer tumors will show up on an x-ray or CT scan and can be measured with a ruler. Blood tests, including those that measure organ function can be performed. A tumor marker test can be done for certain cancers.

Regardless of the test used, whether blood test, cell count, or tumor marker test, it is repeated at specific intervals so that the results can be compared to earlier tests of the same type.

Other measures of the efficacy of cancer treatment include intervals of overall survival (that is time to death from any cause, measured from diagnosis or from initiation of the treatment being evaluated)), cancer-free survival (that is, the length of time after a complete response cancer remains undetectable), and progression-free survival (that is, the length of time after disease stabilization or partial response that resumed tumor growth is not detectable).

In some embodiments, a treatment-induced increase in CPS score is seen. CPS score at baseline may correlate with responsiveness to treatment regimens containing anti-PD-1 or anti-PD-L1 agents. More specifically, CPS score may correlate with improvements in ORR, PFS, or OS when combined with chemotherapy in gastric cancer as exemplified herein.

In some embodiments, the subject has a CPS score. In some embodiments, the CPS score is less than 1. In some embodiments, the CPS score is greater than or equal to 1 and less than 5. In some embodiments, the CPS is greater than or equal to 5.

In some embodiments, treatment induces an increase in CPS score. Changes in CPS score may reflect increased anti-tumor immune activity and may correlate with clinical responses. Changes in CPS may predict clinical response prior to radiological assessment.

In some embodiments, treatment induces an increase in CD8 cells. CD8 T cells are known to mediate anti-tumor immunity and increased CD8 T cells may correlate with clinical responses.

In some embodiments, treatment induces an increase in CD39 together with an increase in CD8 T cells. Expression of CD39 on CD8 T cells is known to enrich for tumor reactive T Cells, differentiating them from bystander T cells.

TABLE S
Sequences.

| SEQ | | | |
|---|---|---|---|
| ID NO: | Region | Binds | Sequence |
| 1 | CDR-H1 | CD39 | SYEMH |
| 2 | CDR-H1 | PD-1 | HYGMN |
| | | (budigalimab) | |
| 3 | CDR-H1 | PD-1 (Nivolumab) | NSGMH |
| 4 | CDR-H1 | PD-1 | NYYMY |
| | | (Pembrolizumab) | |
| 5 | CDR-H2 | CD39 | RINPSVGSTWYAQKFQG |
| 6 | CDR-H2 | PD-1 | WVNTYTGEPTYADDFKG |
| | | (budigalimab) | |
| 7 | CDR-H2 | PD-1 (Nivolumab) | VIWYDGSKRYYADSVKG |
| 8 | CDR-H2 | PD-1 | GINPSNGGTNFNEKFKN |
| | | (Pembrolizumab) | |
| 9 | CDR-H3 | CD39 | GKREGGTEYLRK |
| 10 | CDR-H3 | PD-1 | EGEGLGFGD |
| | | (budigalimab) | |
| 11 | CDR-H3 | PD-1 (Nivolumab) | NDDY |
| 12 | CDR-H3 | PD-1 | RDYRFDMGFDY |
| | | (Pembrolizumab) | |
| 13 | CDR-L1 | CD39 | RASQSVASSYLA |
| 14 | CDR-L1 | PD-1 | RSSQSIVHSHGDTYLE |
| | | (budigalimab) | |
| 15 | CDR-L1 | PD-1 (Nivolumab) | RASQSVSSYLA |
| 16 | CDR-L1 | PD-1 | RASKGVSTSGYSYLH |
| | | (Pembrolizumab) | |
| 17 | CDR-L2 | CD39 | GASNRHT |
| 18 | CDR-L2 | PD-1 | KVSNRFS |
| | | (budigalimab) | |
| 19 | CDR-L2 | PD-1 (Nivolumab) | DASNRAT |
| 20 | CDR-L2 | PD-1 | LASYLES |
| | | (Pembrolizumab) | |
| 21 | CDR-L3 | CD39 | QQYHNAIT |
| 22 | CDR-L3 | PD-1 | FQGSHIPVT |
| | | (budigalimab) | |
| 23 | CDR-L3 | PD-1 (Nivolumab) | QQSSNWPRT |
| 24 | CDR-L3 | PD-1 | QHSRDLPLT |
| | | (Pembrolizumab) | |
| 25 | VH | CD39 | QVQLVQSGAEVKKPGASVKVSCKASGYT |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRKWG |
| | | | QGTLVTVSS |
| 26 | VH | PD-1 | EIQLVQSGAEVKKPGSSVKVSCKASGYT |
| | | (budigalimab) | FTHYGMNWVRQAPGQGLEWVGvNNTYTG |
| | | | EPTYADDFKGRLTFTLDTSTSTAYMELS |
| | | | SLRSEDTAVYYCTREGEGLGFGDWGQGT |
| | | | TVTVSS |
| 27 | VH | PD-1 (Nivolumab) | QVQLVESGGGWQPGRSLRLDCKASGITF |
| | | | SNSGMHWVRQAPGKGLEWVAVIWYDGSK |
| | | | RYYADSVKGRFTISRDNSKNTLFLQMNS |
| | | | LRAEDTAVYYCATNDDYWGQGTLVTVSS |
| 28 | VH | PD-1 | QVQLVQSGVEVKKPGASVKVSCKASGYT |
| | | (Pembrolizumab) | FTNYYMYWVRQAPGQGLEWMGGINPSNG |
| | | | GTNFNEKFKNRVTLTTDSSTTTAYMELK |
| | | | SLQFDDTAVYYCARRDYRFDMGFDYWGQ |
| | | | GTTVTVSS |
| 29 | VL | CD39 | EIVLTQSPGTLSLSPGERATLSCRASQS |
| | | | VASSYLAWYQQKPGQAPRLLIYGASNRH |
| | | | TGIPDRFSGSGSGTDFTLTISRLEPEDF |
| | | | AVYYCQQYHNAITFGGGTKVEIK |
| 30 | VL | PD-1 | DVVMTQSPLSLPVTPGEPASISCRSSQS |
| | | (budigalimab) | IVHSHGDTYLEWYLQKPGQSPQLLIYKV |
| | | | SNRFSGVPDRFSGSGSGTDFTLKISRVE |
| | | | AEDVGVYYCFQGSHIPVTFGQGTKLEIK |
| 31 | VL | PD-1 (Nivolumab) | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQSSNWPRTFGQGTKVEIK |
| 32 | VL | PD-1 | EIVLTQSPATLSLSPGERATLSCRASKG |
| | | (Pembrolizumab) | VSTSGYSYLHWYQQKPGQAPRLLIYLAS |
| | | | YLESGVPARFSGSGSGTDFTLTISSLEP |
| | | | EDFAVYYCQHSRDLPLTFGGGTKVEIK |
| 33 | HC | CD39 | QVQLVQSGAEVKKPGASVKVSCKASGYT |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRKWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 34 | LC | CD39 | EIVLTQSPGTLSLSPGERATLSCRASQS |
| | | | VASSYLAWYQQKPGQAPRLLIYGASNRH |
| | | | TGIPDRFSGSGSGTDFTLTISRLEPEDF |
| | | | AVYYCQQYHNAITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 35 | HC | PD-1 | EIQLVQSGAEVKKPGSSVKVSCKASGYT |
| | | (budigalimab) | FTHYGMNWVRQAPGQGLEWVGWVNTYTG |
| | | | EPTYADDFKGRLTFTLDTSTSTAYMELS |
| | | | SLRSEDTAVYYCTREGEGLGFGDWGQGT |
| | | | TVTVSSASTKGPSVFPLAPSSKSTSGGT |
| | | | AALGCLVKDYFPEPVTVSWNSGALTSGV |
| | | | HTFPAVLQSSGLYSLSSVVTVPSSSLGT |
| | | | QTYICNVNHKPSNTKVDKKVEPKSCDKT |
| | | | HTCPPCPAPEAAGGPSVFLFPPKPKDTL |
| | | | MISRTPEVTCVVVDVSHEDPEVKFNWYV |
| | | | DGVEVHNAKTKPREEQYNSTYRVVSVLT |
| | | | VLHQDWLNGKEYKCKVSNKALPAPIEKT |
| | | | ISKAKGQPREPQVYTLPPSREEMTKNQV |
| | | | SLTCLVKGFYPSDIAVEWESNGQPENNY |
| | | | KTTPPVLDSDGSFFLYSKLTVDKSRWQQ |
| | | | GNVFSCSVMHEALHNHYTQKSLSLSPGK |
| 36 | HC | PD-1 | EIQLVQSGAEVKKPGSSVKVSCKASGYT |
| | | (budigalimab) | FTHYGMNWVRQAPGQGLEWVGWVNTYTG |
| | | | EPTYADDFKGRLTFTLDTSTSTAYMELS |
| | | | SLRSEDTAVYYCTREGEGLGFGDWGQGT |
| | | | TVTVSSASTKGPSVFPLAPSSKSTSGGT |
| | | | AALGCLVKDYFPEPVTVSWNSGALTSGV |
| | | | HTFPAVLQSSGLYSLSSWTVPSSSLGTQ |
| | | | TYICNVNHKPSNTKVDKKVEPKSCDKTH |
| | | | TCPPCPAPEAAGGPSVFLFPPKPKDTLM |
| | | | ISRTPEVTCWVDVSHEDPEVKFNWYVDG |
| | | | VEVHNAKTKPREEQYNSTYRWSVLTVLH |
| | | | QDWLNGKEYKCKVSNKALPAPIEKTISK |
| | | | AKGQPREPQVYTLPPSREEMTKNQVSLT |
| | | | CLVKGFYPSDIAVEWESNGQPENNYKTT |
| | | | PPVLDSDGSFFLYSKLTVDKSRWQQGNV |
| | | | FSCSVMHEALHNHYTQKSLSLSPG |
| 37 | LC | PD-1 | DVVMTQSPLSLPVTPGEPASISCRSSQS |
| | | (budigalimab) | IVHSHGDTYLEWYLQKPGQSPQLLIYKV |
| | | | SNRFSGVPDRFSGSGSGTDFTLKISRVE |
| | | | AEDVGVYYCFQGSHIPVTFGQGTKLEIK |
| | | | RTVAAPSVFIFPPSDEQLKSGTASWCLL |
| | | | NNFYPREAKVQWKVDNALQSGNSQESVT |
| | | | EQDSKDSTYSLSSTLTLSKADYEKHKVY |
| | | | ACEVTHQGLSSPVTKSFNRGEC |
| 38 | HC | PD-1 (Nivolumab) | QVQLVESGGGWQPGRSLRLDCKASGITF |
| | | | SNSGMHWVRQAPGKGLEWVAVIWYDGSK |
| | | | RYYADSVKGRFTISRDNSKNTLFLQMNS |
| | | | LRAEDTAVYYCATNDDYWGQGTLVTVSS |
| | | | ASTKGPSVFPLAPCSRSTSESTAALGCL |
| | | | VKDYFPEPVTVSWNSGALTSGVHTFPAV |
| | | | LQSSGLYSLSSWTVPSSSLGTKTYTCNV |
| | | | DHKPSNTKVDKRVESKYGPPCPPCPAPE |
| | | | FLGGPSVFLFPPKPKDTLMISRTPEVTC |
| | | | WVDVSQEDPEVQFNWYVDGVEVHNAKTK |
| | | | PREEQFNSTYRWSVLTVLHQDWLNGKEY |
| | | | KCKVSNKGLPSSIEKTISKAKGQPREPQ |
| | | | VYTLPPSQEEMTKNQVSLTCLVKGFYPS |
| | | | DIAVEWESNGQPENNYKTTPPVLDSDGS |
| | | | FFLYSRLTVDKSRWQEGNVFSCSVMHEA |
| | | | LHNHYTQKSLSLSLGK |
| 39 | LC | PD-1 (Nivolumab) | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQSSNWPRTFGQGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 40 | HC | PD-1 | QVQLVQSGVEVKKPGASVKVSCKASGYT |
| | | (Pembrolizumab) | FTNYYMYWVRQAPGQGLEWMGGINPSNG |
| | | | GTNFNEKFKNRVTLTTDSSTTTAYMELK |
| | | | SLQFDDTAVYYCARRDYRFDMGFDYWGQ |
| | | | GTTVTVSSASTKGPSVFPLAPCSRSTSE |
| | | | STAALGCLVKDYFPEPVTVSWNSGALTS |
| | | | GVHTFPAVLQSSGLYSLSSVVTVPSSSL |
| | | | GTKTYTCNVDHKPSNTKVDKRVESKYGP |
| | | | PCPPCPAPEFLGGPSVFLFPPKPKDTLM |
| | | | ISRTPEVTCVVVDVSQEDPEVQFNWYVD |
| | | | GVEVHNAKTKPREEQFNSTYRVVSVLTV |
| | | | LHQDWLNGKEYKCKVSNKGLPSSIEKTI |
| | | | SKAKGQPREPQVYTLPPSQEEMTKNQVS |
| | | | LTCLVKGFYPSDIAVEWESNGQPENNYK |
| | | | TTPPVLDSDGSFFLYSRLTVDKSRWQEG |
| | | | NVFSCSVMHEALHNHYTQKSLSLSLGK |
| 41 | LC | PD-1 | EIVLTQSPATLSLSPGERATLSCRASKG |
| | | (Pembrolizumab) | VSTSGYSYLHWYQQKPGQAPRLLIYLAS |
| | | | YLESGVPARFSGSGSGTDFTLTISSLEP |
| | | | EDFAVYYCQHSRDLPLTFGGGTKVEIKR |
| | | | TVAAPSVFIFPPSDEQLKSGTASVVCLL |
| | | | NNFYPREAKVQWKVDNALQSGNSQESVT |
| | | | EQDSKDSTYSLSSTLTLSKADYEKHKVY |
| | | | ACEVTHQGLSSPVTKSFNRGEC |

### EXAMPLES

### Example 1: Inclusion and Exclusion and Dose Identification

Inclusion criteria included male or female subjects ≥18 years of age at the time of screening (or for South Korea: ≥19 years of age at the time of screening), fresh and/or archival tumor tissue, weight ≥35 kg, evidence of measurable disease by computed tomography (CT), CT-positron emission tomography (PET), or magnetic resonance imaging (MM) per Response Evaluation Criteria in Solid Tumors (RECIST) v1.1, a life expectancy >12 weeks, and an Eastern Cooperative Oncology Group (ECOG) performance status score of 0 or 1.

Inclusion criteria also included adequate organ and marrow function, defined as:
a. Absolute neutrophil count ≥1.5 k/μL, platelets ≥75 k/μL (platelets ≥100 k/μL for chemotherapy arms), hemoglobin ≥8 g/dL (hemoglobin ≥9 g/dL for chemotherapy arms).
b. Serum creatinine ≤1.5× upper limit of normal (ULN) or creatinine clearance (CrCl) ≥40 mL/min. NOTE: Subjects eligible for cohort 10 may have CrCl<40 mL/min after approval by the medical monitor.
c. Aspartate aminotransferase (AST)/alanine aminotransferase (ALT) ≤2.5× ULN (or ≤5× ULN with hepatic metastases).
d. Total bilirubin ≤2× ULN (or ≤3× ULN with Gilbert's syndrome).
e. Prothrombin time (PT)/international normalized ratio (INR) and activated partial thromboplastin time (aPTT) ≤1.2× ULN; fibrinogen ≥150 mg/dL.
f. Serum albumin ≥3.0 g/dL.

Subjects also had to have at least 14 days since last dose of chemotherapy or biological therapy or tyrosine kinase inhibitor or high-dose (e.g., >10 mg prednisone or equivalent per day) steroid therapy prior to the loading dose/first dose of study treatment or other second-generation hormone therapy prior to the loading dose/first dose of study treatment. Resolution of adverse effects from any prior chemotherapy, immunotherapy, or prior systemic anticancer therapy, radiotherapy, or surgery to Grade 1 or baseline (except Grade 2 alopecia and Grade 2 sensory neuropathy) were also required. Subjects with a history of congestive heart failure were required to have a cardiac echocardiogram or multigated acquisition scan indicating left ventricular ejection fraction ≥45% within 21 days prior to the loading dose/first dose of study treatment.

Inclusion criteria also included histologically or cytologically confirmed diagnosis of advanced unresectable or metastatic adenocarcinoma of the stomach or gastroesophageal junction with HER2-negative disease (HER2 0 or 1 by IHC or HER2 2+ by IHC and no HER2 gene amplification by in situ hybridization [ISM]). Subjects had to have histologically or cytologically confirmed HER2-negative disease (HER2 0 or 1 by IHC or HER2 2+ by IHC and no HER2 gene amplification by ISH) on primary or metastatic tumor. No prior treatment for metastatic disease and no prior (neo-)adjuvant therapy within 6 months of study enrollment were required.

Exclusion criteria included a history of allergy or hypersensitivity to the study treatment components. Subjects with a history of severe hypersensitivity reaction to any monoclonal antibody (defined as any Grade 3 reaction lasting ≥48 hours despite optimal therapy) were excluded. Subjects that used an investigational agent within fourteen (14) days prior to the loading dose/first dose of study treatment and throughout the study, chemotherapy, radiation therapy, biologic therapy, herbal therapy, or any investigational therapy within 14 days prior to the loading dose/first dose of study treatment were excluded. Palliative radiation therapy to non-target lesions was allowed.

Subjects were excluded that received high-dose (e.g., >10 mg prednisone or equivalent per day) systemic steroid therapy or any other form of immunosuppressive therapy within 14 days prior to the loading dose/first dose of study treatment; however, inhaled, intranasal, intraocular, topical, and intraarticular steroids are allowed. Transient steroid administration as anti-emetic or chemotherapy pre-conditioning (e.g., for paclitaxel) was also allowed. Subjects that received therapeutic anticoagulation were excluded. Prophylactic anticoagulation with low-mol ecul ar-weight heparin, Factor Xa inhibitors, and low-dose aspirin was allowed. Patients with a history of autoimmune disease (e.g., rheumatoid arthritis, systemic lupus erythematosus, inflammatory bowel disease) requiring systemic treatment or transplant that requires systemic steroids or immunosuppressive agents within the last 2 years were excluded. Patients with a history of vitiligo, autoimmune thyroiditis, or mild psoriasis were allowed.

Subjects with a known history of HIV or other chronic immunodeficiency were excluded. Also, subjects with an uncontrolled intercurrent illness were excluded, the illnesses including, but not limited to:
Uncontrolled diabetes.
Ongoing or active bacterial, viral, or fungal infection requiring systemic treatment.
Clinically significant congestive heart failure defined by New York Heart Association Class 3 or Class 4.
Unstable angina, arrhythmia, or myocardial infarction within 6 months prior to screening.
Uncontrolled tumor-related pain. (Subjects requiring narcotic pain medication had to be on a stable regimen at study entry).
Poorly controlled hypertension, defined as a blood pressure consistently above 150/90 mmHg despite optimal medical management.
Uncontrolled pleural effusion, pericardial effusion, or ascites requiring repeated drainage more than once every 28 days. Indwelling drainage catheters (e.g., PleurX®) were allowed.
Active or chronic viral hepatitis B or C infection. Subjects who were positive for hepatitis B surface antigen or hepatitis C antibody were excluded. If hepatitis B core antibody was positive, the subject had to have a negative PCR result before enrollment. Those who are PCR positive were excluded.
Uncontrolled thyroid disease.
Known history of active tuberculosis.
Active infection requiring systemic therapy (Grade ≥2) for more than 3 days within 1 week of dosing.

Subjects with active or untreated central nervous system metastases were excluded. Subjects with brain metastases were eligible provided they were able to show clinical and radiographic SD for at least 4 weeks after definitive therapy and had not used steroids (>10 mg/day of prednisone or equivalent) for at least 4 weeks prior to the loading dose/first dose of study treatment. Subjects with a history of any other malignancy within the past 3 years were excluded (except for successfully treated non-melanoma skin cancer or localized carcinoma in situ that is considered cured or adequately treated by the investigator). Subjects with completely resected cutaneous melanoma (early stage), basal cell carcinoma, cutaneous squamous cell carcinoma, cervical carcinoma in-situ, breast carcinoma in-situ, and localized prostate cancer were eligible.

Women who were pregnant or breastfeeding were excluded. Subjects that received a live vaccine within 28 days prior to the loading dose/first dose of study drug were excluded. Subjects with a history of (except in the setting of PD-(L)1 therapy) or ongoing pneumonitis or interstitial lung disease and history of idiopathic pulmonary fibrosis, organizing pneumonia, bronchiolitis obliterans, drug-induced pneumonitis, or idiopathic pneumonitis were excluded. Subjects who had been previously treated with an anti PD-(L)1 targeting agent were excluded if they had any of the following during the course of their therapy:
a. Any immune-mediated toxicity of Grade 3 or worse severity.
b. Any ocular or neurologic toxicity.
c. Any hypersensitivity to PD-(L)1 targeting agents for subjects in the budigalimab and pembrolizumab-containing cohorts.

Subjects judged by the investigator to have evidence of ongoing hemolysis on hemolysis panel (total, direct and unconjugated serum bilirubin, peripheral blood smear, D-dimers, and serum haptoglobin) were excluded. Subjects that had major surgery per the investigator within 28 days prior to the loading dose/first dose of study drug, and the surgical wound is not fully healed were excluded. A diagnostic or research biopsy did not exclude subjects from enrollment and placement of a vascular access device such as a Port-A-Cath was not considered major surgery. Subjects with a history of major immunologic reaction (Grade 3-4) to any IgG-containing agent were excluded. Subjects with a history of primary immunodeficiency, bone marrow transplantation, chronic lymphocytic leukemia, solid organ transplantation, or previous clinical diagnosis of tuberculosis were excluded. Subjects with a history of Stevens-Johnson syndrome (SJS), Toxic epidermal necrolysis (TEN), or drug reaction with eosinophilia and systemic symptoms (DRESS) were excluded.

Exclusion criteria also included an inability to receive a port or peripherally inserted central catheter, a known hypersensitivity to 5-FU, oxaliplatin, or other platinum agents, a known hypersensitivity to mFOLFOX6 or any of its excipients, known dihydropyrimidine dehydrogenase deficiency (testing not required), and a baseline peripheral neuropathy/paresthesia Grade >1.

6 subjects with various advanced solid tumors received the following combination regimen:
anti-human CD39 antibody (40 mg/kg 7 days prior to Cycle 1 Day 1 followed by 20 mg/kg Q2W).
Budigalimab (500 mg every 4 weeks [Q4W]).
mFOLFOX6 (oxaliplatin 85 mg/m² IV with leucovorin 400 mg/m² IV over 2 hours plus 5-FU 400 mg/m² IV bolus and 2400 mg/m² continuous infusion over 46 hours Q2W).

### Example 2: Anti human CD39 Antibody in Combination with an Anti Human PD-1 Antibody and Chemotherapy in Subjects with Advanced Gastroesophageal Cancer

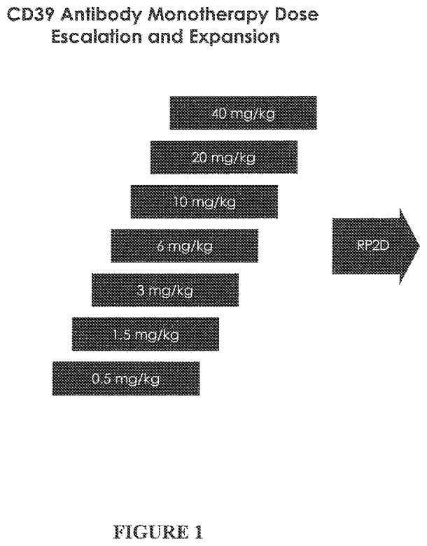

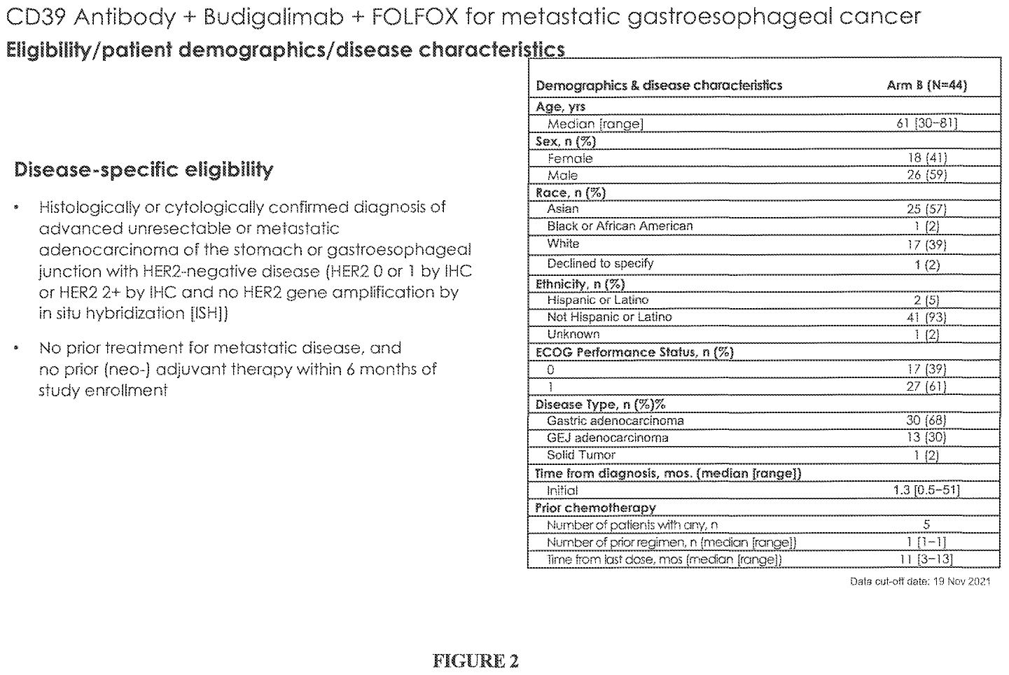

FIG. 1 shows monotherapy dose escalation and expansion and study design for treatment with a CD39 antibody, a PD-1 antibody (budigalimab), and FOLFOX for metastatic gastroesophageal cancer. FIG. 2 shows eligibility and patient demographics and disease characteristics for treatment with a CD39 antibody, budigalimab, and FOLFOX for metastatic gastroesophageal cancer.

70 response-evaluable subjects with human epidermal growth factor receptor 2 (HER2)-negative metastatic gastroesophageal cancer (GEC) were administered at the doses set forth below.
1. Arm A: anti-human CD39 antibody+mFOLFOX6 (n=6).
2. Cohort 3 Arm B: anti-human CD39 antibody+budigalimab+mFOLFOX6 (n=40).
3. Budigalimab+mFOLFOX6 (n=24).

Inclusion criteria included histologically or cytologically confirmed diagnosis of advanced unresectable or metastatic adenocarcinoma of the stomach or gastroesophageal junction with HER2-negative disease (HER2 0 or 1 by IHC or HER2 2+ by IHC and no HER2 gene amplification by in situ hybridization [ISM]). Subjects were required to have histologically or cytologically confirmed HER2-negative disease (HER2 0 or 1 by IHC or HER2 2+ by IHC and no HER2 gene amplification by ISH) on primary or metastatic tumor. Subjects were also required to have no prior treatment for metastatic disease, and no prior (neo-)adjuvant therapy within 6 months of study enrollment.

An RP2D of the anti human CD39 antibody was determined to be a 40 mg/kg loading dose 7 days prior to Cycle 1 Day 1 and then either 30 mg/kg Q3W or 20 mg/kg Q2W. Doses up to 40 mg/kg were determined to be well tolerated as set forth below.

PK demonstrated that a dose of 40 mg/kg Q3W achieved a minimal functional trough level based on activity of anti-human CD39 antibody in preclinical human functional assays (i.e., enhancement of IL-2 from PBMCs and inflammasome activation). The half-life was 18.9 days. After the completion of the dose escalation portion of the study, the totality of the PK data was used for PK modeling for the RP2D. Based on simulations, it was determined that a loading dose of 40 mg/kg one week prior to Cycle 1, Day 1 dosing of 30 mg/kg Q3W or 20 mg/kg Q2W will provide a minimal functional trough level in greater than 80% of subjects before Cycle 1, Day 1 and in cycles thereafter.

Based on the observed safety, PK, pharmacodynamics, and efficacy data, the RP2D of the anti-human PD-1 antibody was determined to be flat doses of 250 mg Q2W, 375 mg Q3W, or 500 mg Q4W. The RP2D was also supported by population PK modeling and simulations that indicated the exposures achieved with 250 mg Q2W, 375 mg Q3W, or 500 mg Q4W doses would result in saturation of PD-1 positive CD4 central memory T-cells and significant PD-L1 blockade, with no further impact on safety.

mFOLFOX6 was administered based on standard clinical practice (oxaliplatin 85 mg/m² IV with leucovorin 400 mg/m² IV over 2 hours plus 5-FU 400 mg/m² IV bolus and 2400 mg/m² continuous infusion over 46 hours, administered Q2W). A change to mFOLFOX6 (i.e., dose reduction or combination modification) may be permitted according to institutional guidelines.

The anti-human CD39 antibody and the anti-human PD-1 antibody were administered over at least 60 minutes during, for example, a 28 day cycle. mFOLFOX6 was administered over 48 hours.

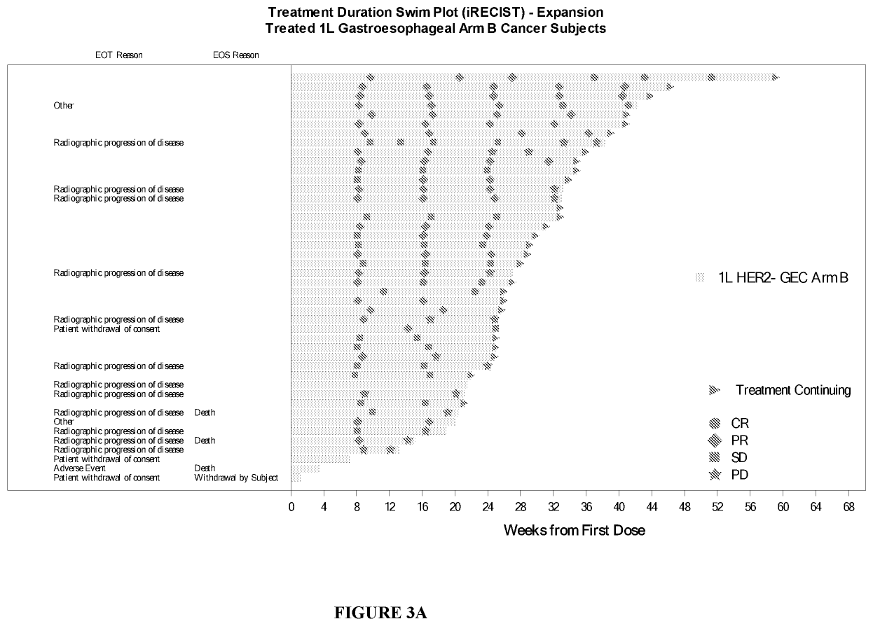

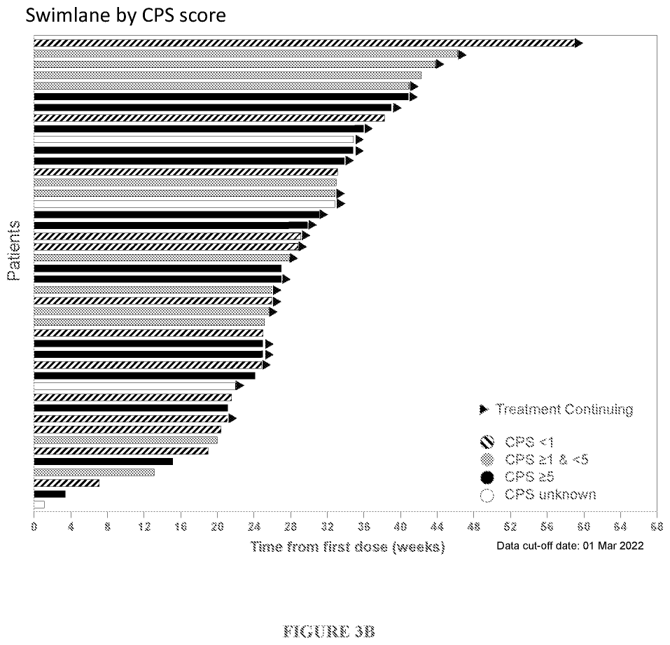

FIG. 3A shows patient response after treatment with a CD39 antibody, budigalimab, and FOLFOX for metastatic gastroesophageal cancer, specifically a treatment duration swim plot and FIG. 3B shows patient response after treatment with a CD39 antibody, budigalimab, and FOLFOX for metastatic gastroesophageal cancer, specifically swimlane by CPS score.

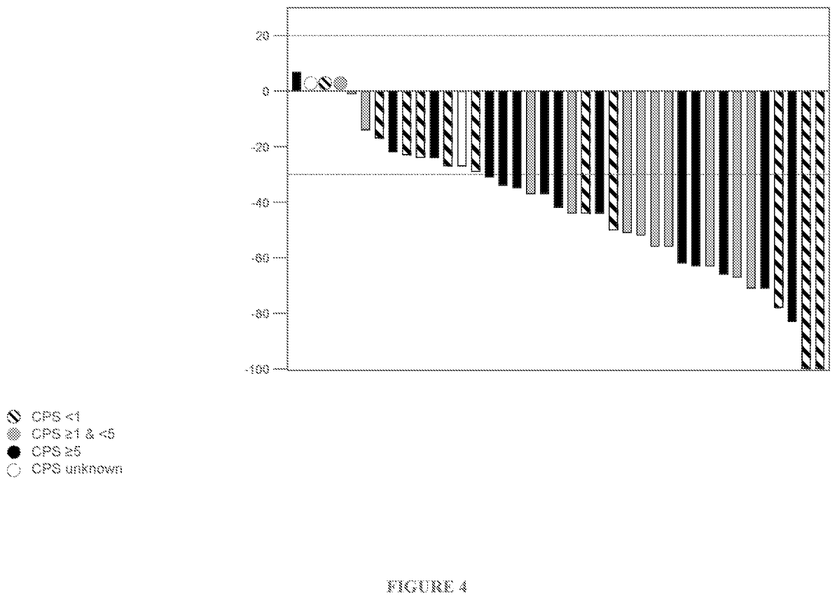

FIG. 4 shows maximum change from baseline for treatment with a CD39 antibody, budigalimab, and FOLFOX for metastatic gastroesophageal cancer, as well as a waterfall plot by CPS score. FIG. 4 shows tumor responses across different PD-L1 levels at baseline.

BOR (RECIST) criteria for a total evaluable population N=40 are shown in Table 1.

TABLE 1
BOR (RECIST)
Total Evaluable Population N = 40 [n (%)]

| | Incl. Unconfirmed | Confirmed |
|---|---|---|
| ORR | 25 (62.5) | 21 (52.5) |
| CR | 4 (10) | 4 (10) |
| PR | 21 (52.5) | 17 (42.5) |
| SD | 12 (30) | 16 (40) |
| PD | 3 (7.5) | 3 (7.5) |
| DCR | 37 (92.5) | 37 (92.5) |

ORR (RECIST) criteria for a subpopulation with known CPS N=37 is shown in Table 2

TABLE 2
ORR (RECIST)
Subpopulation with Known CPS N = 37 [n (%)]

| | Incl. Unconfirmed | Confirmed |
|---|---|---|
| CPS <1 | 5 (45.5) | 4 (36.4) |
| N = 11 | | |
| CPS ≥1 & <5 | 9 (75.0) | 8 (66.7) |
| N = 12 | | |
| CPS ≥5 | 11 (78.6) | 9 (64.3) |
| N = 14 | | |

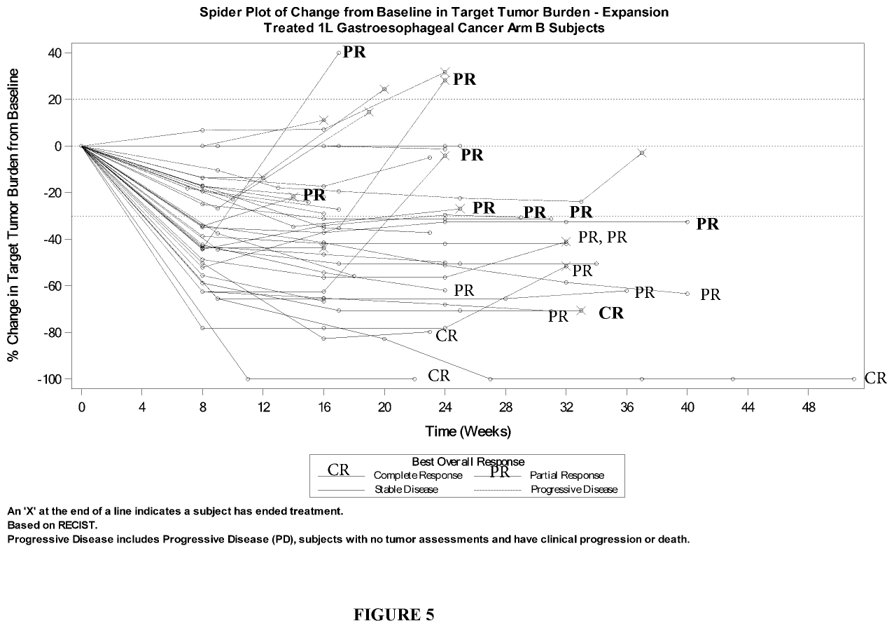

FIG. 5 shows onset of tumor responses for treatment with a CD39 antibody, budigalimab, and FOLFOX for metastatic gastroesophageal cancer.

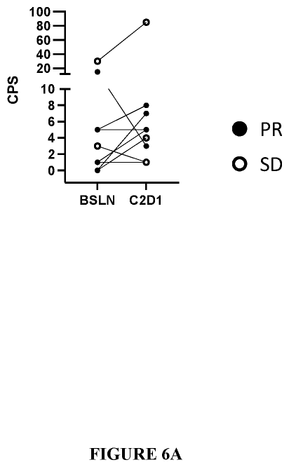

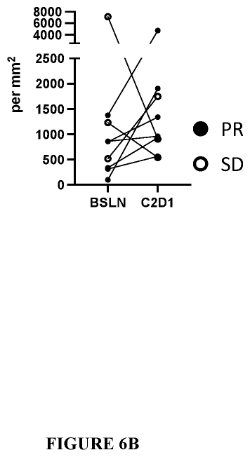

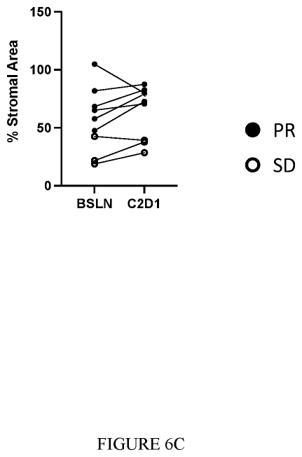

FIG. 6A shows combination treatment leads to an increase in PD-L1 CPS score and CD8 T cells. FIG. 6B shows combination treatment leads to an increase in CD8 cells. FIG. 6C shows combination treatment impacts CD39 stromal treatment.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method for treating a subject suffering from gastric cancer, comprising the step of administering to the subjecta pharmaceutical composition comprising:
i) an anti human CD39 antibody consisting of the heavy chain amino acid SEQ ID NO: 33 and light chain amino acid SEQ ID NO: 34;
ii) an anti-human PD-1 antibody consisting of the heavy chain amino acid SEQ ID NO: 35 and the light chain amino acid SEQ ID NO: 36; and
iii) mFOLFOX6 consisting of oxaliplatin, leucovorin, and fluorouracil (5FU).

2. The method according to claim 1, wherein the anti-human CD39 antibody is administered intravenously at a dosage from 0.5 mg/kg to 40.0 mg/kg.

3. The method according to claim 2, wherein the anti-human CD39 antibody is administered intravenously at a dosage of 20.0 mg/kg.

4. The method according to claim 1, wherein the anti-human PD-1 antibody is administered intravenously at a dosage of 500 mg.

5. The method according to claim 1, wherein mFOLFOX6 is administered intravenously in the following amounts: oxaliplatin at 85 mg/m²; leucovarin at 400 mg/m²; and 5-FU at a bolus infusion of 400 mg/m² and a continuous infusion of 2400 mg/m².

6. The method according to claim 1, wherein the gastric cancer comprises locally advanced or metastatic gastric cancer.

7. The method according to claim 6, wherein the gastric cancer is advanced unresectable or metastatic Her-2− adenocarcinoma of the stomach or gastroesophageal junction.

8. The method according to claim 1, wherein the anti-human CD39 antibody is administered on days 1 and 15 of a 28 day cycle.

9. The method according to claim 1, wherein the mFOLFOX6 is administered on days 1 and 15 of a 28 day cycle.

10. The method according to claim 8, wherein a single loading dose of the anti-human CD39 antibody is administered to the subject at a dosage of 40.0 mg/kg one week before beginning a repetitive portion of a dosing regimen (maintenance doses).

11. The method according to claim 1, wherein the anti-human PD1 antibody is administered to the subject at a dosage of 500 mg every four weeks.

12. The method according to claim 1, wherein the subject has a Partial Response or a Complete Response.

13. The method according to claim 1, wherein the method produces an OS of 20%, 25%, 30%, 35%, 40%, 45%, 50%, 55%, 60%, 65%, 70%, 75%, 80%, 85%, or 90%.

14. The method according to claim 1, wherein the method increases the PFS or DFS.

15. A method for treating a subject suffering from gastric cancer, comprising the step of administering to the subjecta pharmaceutical composition comprising:
i) an anti human CD39 antibody consisting of the heavy chain amino acid SEQ ID NO: 33 and light chain amino acid SEQ ID NO: 34;
ii) an anti-human PD-1 antibody consisting of the heavy chain amino acid SEQ ID NO: 35 and the light chain amino acid SEQ ID NO: 36; and
iii) CAPOX.

16. The method according to claim 15, wherein the anti-human CD39 antibody is administered intravenously at a dosage from 0.5 mg/kg to 40.0 mg/kg.

17. The method according to claim 16, wherein the anti-human CD39 antibody is administered intravenously at a dosage of 20.0 mg/kg.

18. The method according to claim 15, wherein the anti-human PD-1 antibody is administered intravenously at a dosage of 500 mg.

19. The method according to claim 15, wherein the gastric cancer comprises locally advanced or metastatic gastric cancer.

20. The method according to claim 19, wherein the gastric cancer is advanced unresectable or metastatic Her-2— adenocarcinoma of the stomach or gastroesophageal junction.

21. The method according to claim 15, wherein the anti-human CD39 antibody is administered on days 1 and 15 of a 28 day cycle.

22. The method according to claim 15, wherein the anti-human PD1 antibody is administered to the subject at a dosage of 500 mg every four weeks.

23. The method according to claim 15, wherein the subject has a Partial Response or a Complete Response.

24. The method according to claim 15, wherein the method produces an OS of 20%, 25%, 30%, 35%, 40%, 45%, 50%, 55%, 60%, 65%, 70%, 75%, 80%, 85%, or 90%.

25. The method according to claim 15, wherein the method increases the PFS or DFS.

26. The method according to claim 1, wherein the subject has a CPS score that is less than 1, greater than or equal to 1 and less than 5, or greater than or equal to 5.
